(19) United States
(12) Patent Application Publication
(10) Pub. No.: US 2013/0156735 A1
(43) Pub. Date: Jun. 20, 2013

(54) USE OF THE COMBINATION OF TERIFLUNOMIDE AND INTERFERON BETA FOR TREATING MULTIPLE SCLEROSIS

(71) Applicant: SANOFI-AVENTIS U.S LLC, Bridgewater, NJ (US)

(72) Inventors: William BYRNES, Cary, NC (US); Patrice DOUILLET, Paris (FR); Gerald FRANGIN, Paris (FR)

(73) Assignee: SANOFI-AVENTIS U.S LLC, Bridgewater, NJ (US)

(21) Appl. No.: 13/770,238

(22) Filed: Feb. 19, 2013

Related U.S. Application Data
(63) Continuation of application No. 13/346,151, filed on Jan. 9, 2012.
Application No. 13/346,151 is a continuation of application No. PCT/US2010/041274, filed on Jul. 8, 2010.
(60) Provisional application No. 61/225,342, filed on Jul. 14, 2009.

(30) Foreign Application Priority Data
Jul. 10, 2009 (EP) 09305669.5

(51) Int. Cl. A61K 38/21; A61K 31/277

(52) U.S. Cl. CPC A61K 38/215; A61K 31/277
USPC 424/85.6

(57) ABSTRACT

This invention is related to the use of the combination of teriflunomide and interferon beta thereof, for the preparation of a medicament for use in treating multiple sclerosis.

### FIELD OF THE INVENTION

This invention is related to the use of the combination of teriflunomide and interferon beta thereof, for the preparation of a medicament for use in treating multiple sclerosis.

### BACKGROUND OF THE INVENTION

Multiple sclerosis (MS) is a debilitating, inflammatory, neurological illness characterized by demyelination of the central nervous system. The disease primarily affects young adults with a higher incidence in females. Symptoms of the disease include fatigue, numbness, tremor, tingling, dysesthesias, visual disturbances, dizziness, cognitive impairment, urologic dysfunction, decreased mobility, and depression. Four types classify the clinical patterns of the disease: relapsing-remitting, secondary progressive, primary-progressive and progressive-relapsing (S. L. Hauser and D. E. Goodkin, Multiple Sclerosis and Other Demyelinating Diseases in Harrison's Principles of Internal Medicine, 14^{th} Edition, vol. 2, Mc Graw-Hill, 1998, pp. 2409-2419).

The exact etiology of MS is unknown; however, it is strongly suspected that the demyelination characteristic of the disease is the result of an autoimmune response perhaps triggered by an environmental insult, e.g. a viral infection. Specifically, it is hypothesized that MS is caused by a T-cell-mediated, autoimmune inflammatory reaction. The autoimmune basis is strongly supported by the fact that antibodies specific to myelin basic protein (MBP) have been found in the serum and cerebrospinal fluid of MS patients and these antibodies along with T-cells that are reactive to MBP and other myelin proteolipids increase with disease activity. Furthermore, at the cellular level it is speculated that T-cell proliferation and other cellular events, such as activation of B cells and macrophages and secretion of cytokines accompanied by a breakdown of the blood-brain barrier can cause destruction of myelin and oligodendrocytes. (R. A. Adams, M. V. Victor and A. H. Ropper eds, Principles of Neurology, Mc Graw-Hill, New York, 1997, pp.903-921). Progressive MS (primary and secondary) may be based on a neurodegenerative process occurring with demyelination.

The use of (Z)-2-cyano-3-hydroxy-but-2-enoic acid-(4′-trifluoromethylphenyl)-amide (also known as teriflunomide, Formula I) for treating multiple sclerosis has been disclosed in U.S. Pat. No. 6,794,410. Although aforesaid patent discloses that teriflunomide may possibly be combined with another compound known to be effective for treating multiple sclerosis to treat the disease, no specific combination is disclosed to show such efficacy and safety in treating multiple sclerosis.

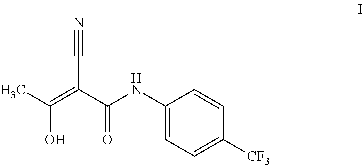 [SMILES: C/C(O)=C(\C#N)C(=O)Nc1ccc(C(F)(F)F)cc1]

### SUMMARY OF THE INVENTION

This invention is related to a method for treating relapsing-remitting form of multiple sclerosis, in a patient in need thereof, comprising administering to the patient about 7 mg or about 14 mg teriflunomide, and a pharmaceutically effective amount of interferon beta-1a or 1b.

### DETAILED DESCRIPTION OF THE INVENTION

As used above, and throughout the description of the invention, the following terms, unless otherwise indicated, shall be understood to have the following meanings:

“Clinically proven effective” mean that the results of clinical trial are statistically significant, i.e., the results of the clinical trial are not likely to be due to chance with an alpha level less than 0.05.

“Patient” means mammals, particularly humans.

“Pharmaceutically effective amount” means an amount of a compound/composition according to the present invention effective in producing the desired therapeutic effect.

“Stable dose of interferon beta-1a or 1b” means administering, for example, about 30 mcg beta-1a once a week, particularly intramuscularly, or about 22 mcg beta-1a three times a week, particularly subcutaneously, or about 44 mcg beta-1a three times a week, particularly subcutaneously, or 0.25 mg beta-1b every other day, particularly subcutaneously.

“Treat” or “treating” means to alleviate symptoms, eliminate the causation of the symptoms either on a temporary or permanent basis, or to prevent or slow the appearance of symptoms of the named disorder or condition.

One particular embodiment of the invention is a method for treating relapsing-remitting form of multiple sclerosis, in a patient in need thereof, comprising administering to the patient about 7 mg or about 14 mg teriflunomide once a day, and a stable dose of interferon beta-1a or 1b.

Another particular embodiment of the invention is a method for treating relapsing-remitting form of multiple sclerosis, in a patient in need thereof, comprising concurrently administering to the patient about 7 mg or about 14 mg teriflunomide once a day, and a stable dose of interferon beta-1a or 1b.

Another particular embodiment of the invention is related to a clinically proven effective method for treating relapsing-remitting form of multiple sclerosis, in a patient in need thereof, comprising administering to the patient about 7 mg or about 14 mg teriflunomide once a day, and a stable dose of interferon beta-1a or 1b.

Another particular embodiment of the invention is related to a method for treating relapsing-remitting form of multiple sclerosis, in a patient in need thereof, comprising administering to the patient about 7 mg teriflunomide once a day and about 30 mcg beta-1a once a week.

Another particular embodiment of the invention is related to a method for treating relapsing-remitting form of multiple sclerosis, in a patient in need thereof, comprising administering to the patient about 7 mg teriflunomide once a day and about 22 mcg beta-1a three times a week.

Another particular embodiment of the invention is related to a method for treating relapsing-remitting form of multiple sclerosis, in a patient in need thereof, comprising administering to the patient about 7 mg teriflunomide once a day and about 44 mcg beta-1a three times a week.

Another particular embodiment of the invention is related to a method for treating relapsing-remitting form of multiple sclerosis, in a patient in need thereof, comprising administering to the patient about 7 mg teriflunomide once a day and about 0.25 mg beta-1b every other day.

Another particular embodiment of the invention is related to a method for treating relapsing-remitting form of multiple sclerosis, in a patient in need thereof, comprising administering to the patient about 14 mg teriflunomide once a day and about 30 mcg beta-1a once a week.

Another particular embodiment of the invention is related to a method for treating relapsing-remitting form of multiple sclerosis, in a patient in need thereof, comprising administering to the patient about 14 mg teriflunomide once a day and about 22 mcg beta-1a three times a week.

Another particular embodiment of the invention is related to a method for treating multiple sclerosis, in a patient in need thereof, comprising administering to the patient about 14 mg teriflunomide once a day and about 44 mcg beta-1a three times a week.

Another particular embodiment of the invention is related to a method for treating relapsing-remitting form of multiple sclerosis, in a patient in need thereof, comprising administering to the patient about 14 mg teriflunomide once a day and about 0.25 mg beta-1b every other day.

Another particular embodiment of the invention is related to a method for treating relapsing-remitting form of multiple sclerosis, wherein teriflunomide is administered orally.

Another particular embodiment of the invention is related to a method for treating relapsing-remitting form of multiple sclerosis, wherein the interferon beta-1a is administered intramuscularly or subcutaneously.

Another particular embodiment of the invention is related to a method for treating relapsing-remitting form of multiple sclerosis, wherein the interferon beta-1b is administered subcutaneously.

Another particular embodiment of the invention is related to a method for reducing the number of T1-Gd lesions in a patient afflicted with relapsing-remitting form of multiple sclerosis, comprising administering to the patient about 7 mg or about 14 mg teriflunomide, and a pharmaceutically effective amount of interferon beta-1a or 1b.

Another particular embodiment of the invention is related to a method for reducing the number of T1-Gd lesions in a patient afflicted with relapsing-remitting form of multiple sclerosis, comprising administering to the patient about 7 mg or about 14 mg teriflunomide once a day, and a stable dose of interferon beta-1a or 1b.

Another particular embodiment of the invention is related to a clinically proven effective method for reducing the number of T1-Gd lesions in patients afflicted with relapsing-remitting form of multiple sclerosis, comprising administering to the patient about 7 mg or about 14 mg teriflunomide once a day, and a stable dose of interferon beta-1a or 1b.

Another particular embodiment of the invention is related to a clinically proven effective method for reducing the number of T1-Gd lesions in patients afflicted with relapsing-remitting form of multiple sclerosis, comprising concurrently administering to the patient about 7 mg or about 14 mg teriflunomide once a day, and a stable dose of interferon beta-1a or 1b.

Another particular embodiment of the invention is related to a method for reducing the number of T1-Gd lesions in a patient afflicted with relapsing-remitting form of multiple sclerosis, comprising administering to the patient about 7 mg or about 14 mg teriflunomide once a day, and a stable dose of interferon beta-1a or 1b, wherein more number of T1-Gd lesions is reduced in the patient than in a patient treated by a stable dose of interferon beta-1a or 1b alone.

Another particular embodiment of the invention is related to a clinically proven effective method for reducing numbers of T1-Gd lesions in patients afflicted with relapsing-remitting form of multiple sclerosis, comprising administering to the patients about 7 mg or about 14 mg teriflunomide once a day, and a stable dose of interferon beta-1a or 1b, wherein more numbers of T1-Gd lesions are reduced in the patients treated by the method than in patients treated by a stable dose of interferon beta-1a or 1b alone.

Another particular embodiment of the invention is related to a method for reducing the number of T1-Gd lesions in patients afflicted with relapsing-remitting form of multiple sclerosis, comprising administering to the patient about 7 mg teriflunomide once a day, and a stable dose of interferon beta-1a or 1b, wherein the number of T1-Gd lesions in the patients is reduced about 82.6% to about 84.6% comparing to the number of lesions in patients treated by a stable dose of interferon beta-1a or 1b alone.

Another particular embodiment of the invention is related to a method for reducing the number of T1-Gd lesions in patients afflicted with relapsing-remitting form of multiple sclerosis, comprising administering to the patient about 14 mg teriflunomide once a day, and a stable dose of interferon beta-1a or 1b, wherein the number of T1-Gd lesions in the patients is reduced about 82.8% to about 84.4% comparing to the number of lesions in patients treated by a stable dose of interferon beta-1a or 1b alone.

Another particular embodiment of the invention is related to a method for reducing the volume of T1-Gd lesions in a patient afflicted with relapsing-remitting form of multiple sclerosis, comprising administering to the patient about 7 mg or about 14 mg teriflunomide, and a pharmaceutically effective amount of interferon beta-1a or 1b.

Another particular embodiment of the invention is related to a method for reducing the volume of T1-Gd lesions in a patient afflicted with relapsing-remitting form of multiple sclerosis, comprising administering to the patient about 7 mg or about 14 mg teriflunomide once a day, and a stable dose of interferon beta-1a or 1b.

Another particular embodiment of the invention is related to a clinically proven effective method for reducing the volume of T1-Gd lesions in patients afflicted with relapsing-remitting form of multiple sclerosis, comprising administering to the patients about 7 mg or about 14 mg teriflunomide once a day, and a stable dose of interferon beta-1a or 1b.

Another particular embodiment of the invention is related to a clinically proven effective method for reducing the volume of T1-Gd lesions in patients afflicted with relapsing-remitting form of multiple sclerosis, comprising concurrently administering to the patients about 7 mg or about 14 mg teriflunomide once a day, and a stable dose of interferon beta-1a or 1b.

Another particular embodiment of the invention is related to a method for reducing the volume of T1-Gd lesions in a patient afflicted with relapsing-remitting form of multiple sclerosis, comprising administering to the patient about 7 mg or about 14 mg teriflunomide once a day, and a stable dose of interferon beta-1a or 1b, wherein more volume of T1-Gd lesions is reduced in the patient treated by the method than in a patient treated by a stable dose of interferon beta-1a or 1b alone.

Another particular embodiment of the invention is related to a clinically proven effective method for reducing the volume of T1-Gd lesions in patients afflicted with relapsing-remitting form of multiple sclerosis, comprising administering to the patients about 14 mg teriflunomide once a day, and a stable dose of interferon beta-1a or 1b, wherein more volumes of T1-Gd lesions are reduced in the patients than in patients treated by a stable dose of interferon beta-1a or 1b alone.

Another particular embodiment of the invention is related to a method for reducing the volume of T1-Gd lesions in patients afflicted with relapsing-remitting form of multiple sclerosis, comprising administering to the patient about 14 mg teriflunomide once a day, and a stable dose of interferon beta-1a or 1b, wherein the volume of T1-Gd lesions in the patients treated by the method is reduced about 64.7% to about 70.6% comparing to the volume of lesions in patients treated by a stable dose of interferon beta-1a or 1b alone.

Another particular embodiment of the invention is related to a method for treating relapsing-remitting form of multiple sclerosis in patients in need thereof comprising administering to the patients 7 mg teriflunomide once a day and a stable dose of interferon-beta 1a or 1b, wherein about 69.4% of the patients are free of T1-Gd lesions after about 24-week treatment.

Another particular embodiment of the invention is related to a method for treating relapsing-remitting form of multiple sclerosis in patients in need thereof comprising administering to the patients 7 mg teriflunomide once a day and a stable dose of interferon-beta 1a or 1b, wherein about 69.4% of the patients are free of T1-Gd lesions after about 48-week treatment.

Another particular embodiment of the invention is related to a method for treating relapsing-remitting form of multiple sclerosis in patients in need thereof comprising administering to the patients 14 mg teriflunomide once a day and a stable dose of interferon-beta 1a or 1b, wherein about 81.6% of the patients are free of T1-Gd lesions after about 24-week treatment.

Another particular embodiment of the invention is related to a method for treating relapsing-remitting form of multiple sclerosis in patients in need thereof comprising administering to the patients 14 mg teriflunomide once a day and a stable dose of interferon-beta 1a or 1b, wherein about 76.3% of the patients are free of T1-Gd lesions after about 48-week treatment.

Another particular embodiment of the invention is related to the use of about 7 mg or about 14 mg teriflunomide for the preparation of a medicament for treating relapsing-remitting form of multiple sclerosis, wherein said medicament is administered to a patient in combination of a pharmaceutically effective amount of interferon beta 1a or 1b.

Another particular embodiment of the invention is related to the use of about 7 mg or about 14 mg teriflunomide for the preparation of a medicament for treating relapsing-remitting form of multiple sclerosis, wherein said medicament is administered once a day to a patient in combination of a stable dose of interferon beta 1a or 1b.

Another particular embodiment of the invention is related to the use of about 7 mg or about 14 mg teriflunomide for the preparation of a clinically proven effective medicament for treating relapsing-remitting form of multiple sclerosis, wherein said medicament is administered once a day in combination of a stable dose of interferon beta 1a or 1b.

Another particular embodiment of the invention is related to the use of about 7 mg or about 14 mg teriflunomide for the preparation of a clinically proven effective medicament for treating relapsing-remitting form of multiple sclerosis, wherein said medicament is administered once a day to a patient who is concurrently on a stable dose of interferon beta 1a or 1b.

Another particular embodiment of the invention is related to the use of about 7 mg or about 14 mg teriflunomide for the preparation of a medicament for reducing the number of T1-Gd lesions in a patient afflicted with relapsing-remitting form of multiple sclerosis, wherein said medicament is administered once a day to the patient in combination of a pharmaceutically effective amount of interferon beta 1a or 1b.

Another particular embodiment of the invention is related to the use of about 7 mg or about 14 mg teriflunomide for the preparation of a medicament for reducing the number of T1-Gd lesions in a patient afflicted with relapsing-remitting form of multiple sclerosis, wherein said medicament is administered once a day to the patient in combination of a stable dose of interferon beta 1a or 1b.

Another particular embodiment of the invention is related to the use of about 7 mg or about 14 mg teriflunomide for the preparation of a clinically proven effective medicament for reducing the number of T1-Gd lesions in a patient afflicted with relapsing-remitting form of multiple sclerosis, wherein said medicament is administered once a day to the patient in combination of a stable dose of interferon beta 1a or 1b.

Another particular embodiment of the invention is related to the use of about 7 mg or about 14 mg teriflunomide for the preparation of a medicament for reducing the number of T1-Gd lesions in a patient afflicted with relapsing-remitting form of multiple sclerosis, wherein said medicament is administered once a day to the patient who is concurrently on a stable dose of interferon beta 1a or 1b.

Another particular embodiment of the invention is related to the use of about 7 mg or about 14 mg teriflunomide for the preparation of a medicament for reducing the number of T1-Gd lesions in a patient afflicted with relapsing-remitting form of multiple sclerosis, wherein said medicament is administered once a day to the patient in combination of a stable dose of interferon beta 1a or 1b such that more number of T1-Gd lesions are reduced in the patient than in a patient treated by a stable dose of interferon beta-1a or 1b alone.

Another particular embodiment of the invention is related to the use of about 7 mg or about 14 mg teriflunomide for the preparation of a clinically proven effective medicament for reducing the number of T1-Gd lesions in patients afflicted with relapsing-remitting form of multiple sclerosis, wherein said medicament is administered once a day to the patients in combination of a stable dose of interferon beta 1a or 1b such that more number of T1-Gd lesions are reduced in the patients than in patients treated by a stable dose of interferon beta-1a or 1b alone.

Another particular embodiment of the invention is related to the use of about 7 mg teriflunomide for the preparation of a medicament for reducing the number of T1-Gd lesions in patients afflicted with relapsing-remitting form of multiple sclerosis, wherein said medicament is administered once a day to the patients in combination of a stable dose of interferon beta 1a or 1b such that the number of T1-Gd lesions in the patients is reduced about 56% to about 84.6% comparing to the number of lesions in patients treated by a stable dose of interferon beta-1a or 1b alone.

Another particular embodiment of the invention is related to the use of about 14 mg teriflunomide for the preparation of a medicament for reducing the number of T1-Gd lesions in patients afflicted with relapsing-remitting form of multiple sclerosis, wherein said medicament is administered once a day to the patients in combination of a stable dose of interferon beta 1a or 1b such that the number of T1-Gd lesions in the patients is reduced about 81% to about 82.8% comparing to the number of lesions in patients treated by a stable dose of interferon beta-1a or 1b alone.

Another particular embodiment of the invention is related to the use of about 7 mg or about 14 mg teriflunomide for the preparation of a medicament for reducing the volume of T1-Gd lesions in a patient afflicted with relapsing-remitting form of multiple sclerosis, wherein said medicament is administered once a day to the patient in combination of a pharmaceutically effective amount of interferon beta 1a or 1b.

Another particular embodiment of the invention is related to the use of about 7 mg or about 14 mg teriflunomide for the preparation of a medicament for reducing the volume of T1-Gd lesions in a patient afflicted with relapsing-remitting form of multiple sclerosis, wherein said medicament is administered once a day to the patient in combination of a stable dose of interferon beta 1a or 1b.

Another particular embodiment of the invention is related to the use of about 7 mg or about 14 mg teriflunomide for the preparation of a clinically proven effective medicament for reducing the volume of T1-Gd lesions in a patient afflicted with relapsing-remitting form of multiple sclerosis, wherein said medicament is administered once a day to the patient in combination with a stable dose of interferon beta 1a or 1b.

Another particular embodiment of the invention is related to the use of about 7 mg or about 14 mg teriflunomide for the preparation of a medicament for reducing the volume of T1-Gd lesions in a patient afflicted with relapsing-remitting form of multiple sclerosis, wherein said medicament is administered once a day to the patient who is concurrently on a stable dose of interferon beta 1a or 1b.

Another particular embodiment of the invention is related to the use of about 7 mg or about 14 mg teriflunomide for the preparation of a medicament for reducing the volume of T1-Gd lesions in a patient afflicted with relapsing-remitting form of multiple sclerosis, wherein said medicament is administered once a day to the patient in combination of a stable dose of interferon beta 1a or 1b such that more volume of T1-Gd lesions are reduced in the patient than in a patient treated by a stable dose of interferon beta-1a or 1b alone.

Another particular embodiment of the invention is related to the use of about 7 mg or about 14 mg teriflunomide for the preparation of a clinically proven effective medicament for reducing the volume of T1-Gd lesions in patients afflicted with relapsing-remitting form of multiple sclerosis, wherein said medicament is administered once a day to the patients in combination of a stable dose of interferon beta 1a or 1b such that more volume of T1-Gd lesions are reduced in the patients than in patients treated by a stable dose of interferon beta-1a or 1b alone.

Another particular embodiment of the invention is related to the use of about 7 mg teriflunomide for the preparation of a medicament for reducing the volume of T1-Gd lesions in patients afflicted with relapsing-remitting form of multiple sclerosis, wherein said medicament is administered once a day to the patients in combination of a stable dose of interferon beta 1a or 1b such that the volume of T1-Gd lesions in the patients is reduced about 46% comparing to the number of lesions in patients treated by a stable dose of interferon beta-1a or 1b alone.

Another particular embodiment of the invention is related to the use of about 14 mg teriflunomide for the preparation of a medicament for reducing the number of T1-Gd lesions in patients afflicted with relapsing-remitting form of multiple sclerosis, wherein said medicament is administered once a day to the patients in combination of a stable dose of interferon beta 1a or 1b such that the number of T1-Gd lesions in the patients is reduced about 66% comparing to the number of lesions in patients treated by a stable dose of interferon beta-1a or 1b alone.

Another particular embodiment of the invention is related to the use of about 7 mg teriflunomide for the preparation of a medicament for treating relapsing-remitting form of multiple sclerosis, wherein said medicament is administered once a day to patients in combination of a stable dose of interferon beta 1a or 1b such that about 70% of the patients are free of T1-Gd lesions after about 24-week treatment.

Another particular embodiment of the invention is related to the use of about 7 mg teriflunomide for the preparation of a medicament for treating relapsing-remitting form of multiple sclerosis, wherein said medicament is administered once a day to patients in combination of a stable dose of interferon beta 1a or 1b such that about 69% of the patients are free of T1-Gd lesions after about 48-week treatment.

Another particular embodiment of the invention is related to the use of about 14 mg teriflunomide for the preparation of a medicament for treating relapsing-remitting form of multiple sclerosis, wherein said medicament is administered once a day to patients in combination of a stable dose of interferon beta 1a or 1b such that about 70% of the patients are free of T1-Gd lesions after about 24-week treatment.

Another particular embodiment of the invention is related to the use of about 14 mg teriflunomide for the preparation of a medicament for treating relapsing-remitting form of multiple sclerosis, wherein said medicament is administered once a day to patients in combination of a stable dose of interferon beta 1a or 1b such that about 76% of the patients are free of T1-Gd lesions after about 48-week treatment.

It is now clinically proved that the addition of teriflunomide to stable-dosed interferon improves disease control beyond interferon alone, in terms of MRI activity, without added concerns regarding safety and tolerability.

### EXAMPLES

The present invention may be better understood by reference to the following non-limiting Examples, which are exemplary of the invention. They should in no way be construed, however, as limiting the broad scope of the invention.

### Example 1

A placebo-controlled, double-blinded, randomized, 3-parallel group stratified study was conducted in relapsing-remitting multiple sclerosis patient who were concurrently on a stable dose of interferon beta. The dose level of interferon beta was defined as follows:
Low dose: AVONEX® (interferon beta-1a) 30 mcg once a week intramuscularly, or REBIF® (interferon beta-1a) 22 mcg three times per week subcutaneously.
High dose: REBIF® 44 mcg three times per week subcutaneously, or BETASERON® (interferon beta-1b) 0.25 mg every other day subcutaneously.

Around 40 patients were treated in each treatment group (placebo: 41, 7 mg: 37, and 14 mg: 38). A third of patients in each group were treated with low dose interferon beta 1a. The demographic and disease baseline characteristics were generally comparable amount the 3 treatment groups. The mean age of the study population was 40.1 years. The majority of patients were female (69.8%) and had a relapsing-remitting type of MS (87.9%), with a diagnosis of MS since around 8 years ago and around 40% of patients had no relapse in the previous year. The base line Expanded Disability Status Scale (EDSS) score was similar between treatment groups (around 2.5).

Method:

Safety was evaluated from Treatment Emergent Adverse Event (TEAE), physical examination and laboratory data (e.g. Liver Function Test, every 2-8 weeks), ECG and abdominal ultrasound (24-weeks). Brain MRI (magnetic resonance image) activity, including T1-gadolinium (T1-Gd) with central reading were recorded every 8-week. Relapses were recorded and EDSS was performed at 24-week.

Results:

Safety: Approximately 90% of patients completed the 24-week treatment period in each group. Treatment was prematurely discontinued for TEAE in one patient in each group. The proportion of patients with TEAE due to increased ALT/SGPT (Alanine Transaminase/Serum Glutamic Pyruvic Transaminase) was higher on 14 mg (28.9%) than on 7 mg (13.5%) or placebo (12.2%). Among them, the proportion of patients with ALT/SGPT greater than 3× ULN (three times above the Upper Limit of Normal range for the central laboratory) was low (4.9% in placebo; 0% in 7 mg; 5.3% in 14 mg) with one treatment discontinuation from placebo and one from 14 mg. There was no case of concomitant bilirubin increase. The proportion of patients with TEAE potentially related to immunosuppression (including white blood cell counts, infections and infestations) was slightly higher in the teriflunomide groups (placebo: 32%, 7 mg: 49%, 14 mg: 50%). Among these events upper respiratory tract infections (nasopharyngitis, sinusitis) appeared to be more frequent at 14 mg (23.7%) than placebo (14.6%) and 7 mg (10.8%). None of these events led to treatment discontinuation.

Efficacy: As shown in Tables 1, 2 and 3, the adjusted results of the 24 week study showed that patients taking 7 mg or 14 mg teriflunomide in addition to stable-dose IFN-beta experienced a significant relative risk reduction in the number of gadolinium enhancing T1 (T1-Gd) lesions on brain magnetic resonance imaging, 82.6% (p=0.0009) and 84.4% (p<0.0001), respectively, compared with placebo added to IFN-beta. There was also a trend to a dose-dependent relative reduction of T1-Gd lesion volume in both teriflunomide arms compared to placebo of 67.6% at 7 mg (p=0.1931) and 64.7% at 14 mg (p=0.0072). The proportion of patients free of T1-Gd lesions was higher in both teriflunomide arms (placebo: 57.9%, 7 mg: 69.4%, 14 mg: 81.6%). A few relapses were reported during the 24-week period (5 on placebo, 5 on 7 mg and 2 on 14 mg).

Conclusion: The addition of teriflunomide to stable-dosed interferon beta significantly improved disease control beyond interferon beta alone, as evaluated by T1-gadolinium

MRI activity, with acceptable safety and tolerability over 24 weeks of treatment.

TABLE 1
Total number of Gd-enhancing T1-lesions per MRI scan - ITT population

| | | | teriflunomide | | | |
|---|---|---|---|---|---|---|
| | | | 7 mg | | 14 mg | |
| | Placebo + IFN-β | | teri + IFN-β | | teri + IFN-β | |
| | (N = 41) | | (N = 37) | | (N = 38) | |
| Baseline | | | | | | |
| Patients with >=1 Gd-enhancing | | | | | | |
| T1-lesions | | | | | | |
| Yes | 9 | (22.5%) | 8 | (21.6%) | 8 | (21.1%) |
| No | 31 | (77.5%) | 29 | (78.4%) | 30 | (78.9%) |
| Patient Gd-enhancing T1-lesions | | | | | | |
| N | 40 | | 37 | | 38 | |
| Mean (SD) | 0.725 | (1.679) | 0.838 | (2.892) | 0.579 | (1.734) |
| Median | 0.000 | | 0.000 | | 0.000 | |
| Min:Max | 0.00:7.00 | | 0.00:17.00 | | 0.00:10.00 | |
| Post-baseline | | | | | | |
| Patients with >=1 Gd-enhancing | | | | | | |
| T1-lesions | | | | | | |
| Yes | 16 | (42.1%) | 11 | (30.6%) | 7 | (18.4%) |
| No | 22 | (57.9%) | 25 | (69.4%) | 31 | (81.6%) |
| P-value^{a} | | | 0.3416 | | 0.0445 | |
| Number of Gd-enhancing T1- | | | | | | |
| lesions | | | | | | |
| 0 | 22 | (57.9%) | 25 | (69.4%) | 31 | (81.6%) |
| 1 | 3 | (7.9%) | 8 | (22.2%) | 4 | (10.5%) |
| 2 | 3 | (7.9%) | 0 | | 1 | (2.6%) |
| 3 | 3 | (7.9%) | 1 | (2.8%) | 1 | (2.6%) |
| >=4 | 7 | (18.4%) | 2 | (5.6%) | 1 | (2.6%) |
| Total number of Gd-enhancing T1- | 84 | | 33 | | 15 | |
| lesions | | | | | | |
| Total number of scans | 113 | | 98 | | 108 | |
| Unadjusted Gd-enhancing T1- | 0.743 | | 0.337 | | 0.139 | |
| lesions per scan^{b} | | | | | | |
| Adjusted Gd-enhancing T1-lesions | | | | | | |
| per scan^{c} | | | | | | |
| Estimate (95% CI) | 0.570 | (0.350, 0.929) | 0.099 | (0.041, 0.241) | 0.089 | (0.050, 0.159) |
| Relative risk (95% CI) | | | 0.174 | (0.062, 0.487) | 0.156 | (0.080, 0.304) |
| P-value | | | 0.0009 | | <.0001 | |
| Patient Gd-enhancing T1-lesions | | | | | | |
| per scan^{d} | | | | | | |
| N | 38 | | 36 | | 38 | |
| Mean (SD) | 0.776 | (1.430) | 0.417 | (1.238) | 0.132 | (0.376) |

| | | | |
|---|---|---|---|
| Median | 0.000 | 0.000 | 0.000 |
| Min:Max | 0.00:5.67 | 0.00:5.67 | 0.00:2.00 |

^{a}From Fisher's exact test.
^{b}The total number of Gd-enhancing T1-lesion that occurred during the study divided by the total number of scans during the study.
^{c}Poisson model with the total number of Gd-enhancing T1-lesions as the response variable, baseline number of Gd-enhancing T1-lesions, treatment, IFN-β dose strata and region as covariates, and log-transformed number of scans as an offset variable.
^{d}The number of Gd-enhancing T1-lesions for each patient divided by the number of scans for hat patient.

TABLE 2
Total volume (ml) of Gd-enhancing T1-lesions per
MRI scan - ITT population

| | | teriflunomide | |
|---|---|---|---|
| | Placebo + | 7 mg | 14 mg |
| | IFN-β | teri + IFN-β | teri + IFN-β |
| | (N = 41) | (N = 37) | (N = 38) |
| Baseline | | | |
| N | 40 | 37 | 38 |
| Mean (SD) | 0.078 (0.201) | 0.107 (0.344) | 0.047 (0.120) |
| Median | 0.000 | 0.000 | 0.000 |
| Min:Max | 0.00:0.94 | 0.00:1.67 | 0.00:0.54 |
| Post-baseline | | | |
| Gd-enhancing | 0.068 | 0.022 | 0.024 |
| T1-lesions per | | | |
| scan^{a} | | | |
| Patient Gd-enhancing | | | |
| T1-lesions per scan^{b} | | | |
| N | 38 | 36 | 38 |
| Mean (SD) | 0.069 (0.154) | 0.037 (0.149) | 0.022 (0.087) |
| Median | 0.000 | 0.000 | 0.000 |
| Min:Max | 0.00:0.67 | 0.00:0.87 | 0.00:0.50 |
| P-value^{c} | | 0.1931 | 0.0072 |

^{a}The total volume of Gd-enhancing T1-lesion that occurred during the study divided by the total number of scans during the study.
^{b}The volume of Gd-enhancing T1-lesions for each patient divided by the number of scans for that patient.
^{c}Rank ANCOVA adjusted for IFN-β dose strata, region and ranked baseline volume of Gd-enhancing T1-lesions.

TABLE 3
Analysis of MS relapse - ITT population

| | | | teriflunomide | | | |
|---|---|---|---|---|---|---|
| | | | 7 mg | | 14 mg | |
| | Placebo + IFN-β | | teri + IFN-β | | teri + IFN-β | |
| | (N = 41) | | (N = 37) | | (N = 38) | |
| Number of patients with >=1 | | | | | | |
| relapses | | | | | | |
| Yes | 5 | (12.2%) | 5 | (13.5%) | 2 | (5.3%) |
| No | 36 | (87.8%) | 32 | (86.5%) | 36 | (94.7%) |
| Number of relapses | | | | | | |
| 0 | 36 | (87.8%) | 32 | (86.5%) | 36 | (94.7%) |
| 1 | 5 | (12.2%) | 5 | (13.5%) | 2 | (5.3%) |
| 2 | 0 | | 0 | | 0 | |
| 3 | 0 | | 0 | | 0 | |
| >=4 | 0 | | 0 | | 0 | |
| Total number of relapses | 5 | | 5 | | 2 | |
| Total patient-years followed | 17.9 | | 16.4 | | 17.3 | |
| Unadjusted annualized relapses | 0.279 | | 0.305 | | 0.116 | |
| rate^{a} | | | | | | |
| Adjusted annualized relapse rate^{b} | | | | | | |
| Estimate (95% CI) | 0.260 | (0.108, 0.625) | 0.280 | (0.101, 0.774) | 0.109 | (0.031, 0.388) |
| Relative risk (95% CI) | | | 1.079 | (0.342, 3.403) | 0.420 | (0.085, 2.063) |
| P-value | | | 0.8968 | | 0.2852 | |
| Patient annualized relapse rate^{c} | | | | | | |
| N | 41 | | 37 | | 38 | |
| Mean (SD) | 0.259 | (0.703) | 0.360 | (0.992) | 0.112 | (0.482) |

| | | | |
|---|---|---|---|
| Median | 0.000 | 0.000 | 0.000 |
| Min:Max | 0.00:2.19 | 0.00:4.62 | 0.00:2.16 |

^{a}The total number of confirmed relapses that occurred during the study divided by the total number of patient-years followed in the study.
^{b}Poisson model with the total number of confirmed relapses onset between randomization date and last dose date as the response variable, treatment, IFN-β dose strata and region as covariates, and log-transformed standardized study duration as an offset variable.
^{c}The number of confirmed relapses for each patient divided by the number of years followed in the study for that patient.

### Example 2

Design/Methods: 86 of the116 patients that were randomized to treatment (placebo: 41; 7 mg: 37; 14 mg: 38) for the first 6-months in Example 1 completed this 6-month period and accepted to continue medication for an additional 6-month period (placebo: 31, 7 mg: 28, 14 mg: 27). Safety was evaluated from TEAE, physical exam (every 6 weeks), laboratory data (every 6 weeks), ECG (at the close-out visit), pancreatic ultrasound (at the close-out visit) and brain MRI (at the close-out visit). Relapses were recorded and EDSS was performed every 6-weeks. Annualized relapse rates (ARR) were estimated by a Poisson model adjusted to IFN strata and to geographical regions. The following results report the entire 48-week double-blind treatment period.

Results: Baseline characteristics were similar among groups: mean age 40 years, 70% female, mean EDSS 2.5. 40% of patients were relapse-free in the previous year and 33% of patients were on low-dose IFN-beta.

Safety: Eight TEAEs led to treatment discontinuation (placebo: 2; 7 mg: 3; 14 mg: 3). Hepatic disorder TEAEs were mainly asymptomatic liver enzyme increases. Six patients had ALT increased above 3ULN (placebo: 2; 7 mg: 1; 14 mg: 3) without a concomitant increase in bilirubin. The most frequently reported treatment emergent adverse events were upper respiratory tract infections as a whole (placebo: 17.1%; 7 mg: 16.2%; 14 mg: 23.7%), mainly nasopharyngitis and sinusitis, all type of headaches (placebo:7.3%; 7 mg: 5.4%; 14 mg: 18.4%), all gastrointestinal disorders (placebo: 24.4%; 7 mg: 18.9%; 14 mg: 31.6%). White blood cell counts decreases were numerically comparable in both teriflunomide and placebo treatment groups (placebo: 3; 7 mg: 3; 14 mg: 4) and no patients discontinued treatment due to neutropenia or infection.

Efficacy: As shown in Tables 4, 5 and 6, the adjusted results from the 48 week of study showed that patients taking 7 mg or 14 mg teriflunomide in addition to stable-dose IFN-beta experienced a significant relative risk reduction in the number of T1-Gd lesions on brain magnetic resonance imaging, 84.6% (p=0.0005) and 82.8% (p<0.0001), for 7 mg and 14 mg when added to IFN-beta respectively, compared with placebo added to IFN-beta. There was a trend to a dose-dependent relative reduction of T1-Gd lesion volume in both teriflunomide arms compared to placebo of 72.1% at 7 mg (p=0.1104) and 70.6% at 14 mg (p=0.0154). There was also a dose-dependent trend to a reduction in annualized relapse rate of 32.6% (p=0.43) and 57.9% (p=0.11) in 7 mg or 14 mg teriflunomide groups respectively compared to IFN-beta. The proportion of patients free of T1-Gd lesions was higher in both teriflunomide arms (placebo: 55.3%; 7 mg: 69.4%; 14 mg: 76.3%).

Conclusion: In this one-year study, the addition of teriflunomide to stable-dosed IFN-beta significantly improved disease control as evaluated by MRI activity beyond IFN-beta alone, and trended to a similar reduction in clinical relapse—all with good safety and tolerability.

TABLE 4
Total number of Gd-enhancing T1-lesions per MRI scan - ITT population

| | | | teriflunomide | | | |
|---|---|---|---|---|---|---|
| | | | 7 mg | | 14 mg | |
| | Placebo + IFN-β | | teri + IFN-β | | teri + IFN-β | |
| | (N = 41) | | (N = 37) | | (N = 38) | |
| Baseline | | | | | | |
| Patients with >=1 Gd-enhancing | | | | | | |
| T1-lesions | | | | | | |
| Yes | 9 | (22.5%) | 8 | (21.6%) | 8 | (21.1%) |
| No | 31 | (77.5%) | 29 | (78.4%) | 30 | (78.9%) |
| Patient Gd-enhancing T1- | | | | | | |
| lesions | | | | | | |
| N | 40 | | 37 | | 38 | |
| Mean (SD) | 0.725 | (1.679) | 0.838 | (2.892) | 0.579 | (1.734) |

| | | | |
|---|---|---|---|
| Median | 0.000 | 0.000 | 0.000 |
| Min:Max | 0.00:7.00 | 0.00:17.00 | 0.00:10.00 |
| Post-baseline | | | |
| Patients with >=1 Gd-enhancing | | | |
| T1-lesions | | | |

| Yes | 17 | (44.7%) | 11 | (30.6%) | 9 | (23.7%) |
|---|---|---|---|---|---|---|
| No | 21 | (55.3%) | 25 | (69.4%) | 29 | (76.3%) |
| P-value^{a} | | | 0.2381 | | 0.0896 | |
| Number of Gd-enhancing T1- | | | | | | |
| lesions | | | | | | |
| 0 | 21 | (55.3%) | 25 | (69.4%) | 29 | (76.3%) |
| 1 | 4 | (10.5%) | 8 | (22.2%) | 6 | (15.8%) |
| 2 | 2 | (5.3%) | 0 | | 1 | (2.6%) |
| 3 | 2 | (5.3%) | 0 | | 1 | (2.6%) |
| >=4 | 9 | (23.7%) | 3 | (8.3%) | 1 | (2.6%) |
| Total number of Gd-enhancing | 101 | | 35 | | 17 | |
| T1-lesions | | | | | | |
| Total number of scans | 144 | | 123 | | 132 | |
| Unadjusted Gd-enhancing T1- | 0.701 | | 0.285 | | 0.129 | |
| lesions per scan^{b} | | | | | | |
| Adjusted Gd-enhancing T1- | | | | | | |
| lesions per scan^{c} | | | | | | |
| Estimate (95% CI) | 0.521 | (0.318, 0.854) | 0.080 | (0.032, 0.204) | 0.090 | (0.052, 0.154) |
| Relative risk (95% CI) | | | 0.154 | (0.053, 0.445) | 0.172 | (0.092, 0.323) |
| P-value | | | 0.0005 | | <.0001 | |
| Patient Gd-enhancing T1- | | | | | | |
| lesions per scan^{d} | | | | | | |
| N | 38 | | 36 | | 38 | |
| Mean (SD) | 0.729 | (1.305) | 0.372 | (1.106) | 0.138 | (0.369) |

| | | | |
|---|---|---|---|
| Median | 0.000 | 0.000 | 0.000 |
| Min:Max | 0.00:4.60 | 0.00:5.00 | 0.00:2.00 |

^{a}From Fisher's exact test.
^{b}The total number of Gd-enhancing T1-lesion that occurred during the study divided by the total number of scans during the study.
^{c}Poisson model with the total number of Gd-enhancing T1-lesions as the response variable, baseline number of Gd-enhancing T1-lesions, treatment, IFN-beta dose stratum, and region as covariates, and log-transformed number of scans as an offset variable.
^{d}The number of Gd-enhancing T1-lesions for each patient divided by the number of scans for that patient.

TABLE 5
Total volume (ml) of Gd-enhancing T1-lesions per
MRI scan - ITT population

| | | teriflunomide | |
|---|---|---|---|
| | Placebo + | 7 mg | 14 mg |
| | IFN-β | teri + IFN-β | teri + IFN-β |
| | (N = 41) | (N = 37) | (N = 38) |
| Baseline | | | |
| N | 40 | 37 | 38 |
| Mean (SD) | 0.078 (0.201) | 0.107 (0.344) | 0.047 (0.120) |
| Median | 0.000 | 0.000 | 0.000 |
| Min:Max | 0.00:0.94 | 0.00:1.67 | 0.00:0.54 |
| Post-baseline | | | |
| Gd-enhancing | 0.068 | 0.019 | 0.02 |
| T1-lesions per scan^{a} | | | |
| Patient Gd-enhancing | | | |
| T1-lesions per scan^{b} | | | |
| N | 38 | 36 | 38 |
| Mean (SD) | 0.067 (0.164) | 0.036 (0.148) | 0.023 (0.087) |
| Median | 0.000 | 0.000 | 0.000 |
| Min:Max | 0.00:0.88 | 0.00:0.87 | 0.00:0.50 |
| P-value^{c} | | 0.1104 | 0.0154 |

^{a}The total volume of Gd-enhancing T1-lesion that occurred during the study divided by the total number of scans during the study.
^{b}The volume of Gd-enhancing T1-lesions for each patient divided by the number of scans for that patient.
^{c}Rank ANCOVA adjusted for IFN-beta dose strata, region and ranked baseline volume of Gd-enhancing T1-lesions.

TABLE 6
Analysis of MS relapse - ITT population

| | | | teriflunomide | | | |
|---|---|---|---|---|---|---|
| | | | 7 mg | | 14 mg | |
| | Placebo + IFN-β | | teri + IFN-β | | teri + IFN-β | |
| | (N = 41) | | (N = 37) | | (N = 38) | |
| Number of patients with | | | | | | |
| >=1 relapses | | | | | | |
| Yes | 8 | (19.5%) | 7 | (18.9%) | 5 | (13.2%) |
| No | 33 | (80.5%) | 30 | (81.1%) | 33 | (86.8%) |
| Number of relapses | | | | | | |
| 0 | 33 | (80.5%) | 30 | (81.1%) | 33 | (86.8%) |
| 1 | 4 | (9.8%) | 6 | (16.2%) | 5 | (13.2%) |
| 2 | 3 | (7.3%) | 1 | (2.7%) | 0 | |
| 3 | 1 | (2.4%) | 0 | | 0 | |
| >=4 | 0 | | 0 | | 0 | |
| Total number of relapses | 13 | | 8 | | 5 | |
| Total patient-years | 31.9 | | 28.1 | | 29.5 | |
| followed | | | | | | |
| Unadjusted annualized | 0.408 | | 0.285 | | 0.169 | |
| relapses rate^{a} | | | | | | |
| Adjusted annualized | | | | | | |
| relapse rate^{b} | | | | | | |
| Estimate (95% CI) | 0.343 | (0.162, 0.727) | 0.231 | (0.101, 0.529) | 0.144 | (0.065, 0.318) |
| Relative risk (95% CI) | | | 0.674 | (0.250, 1.816) | 0.421 | (0.150, 1.182) |
| P-value | | | 0.4355 | | 0.1005 | |
| Patient annualized | | | | | | |
| relapse rate^{c} | | | | | | |
| N | 41 | | 37 | | 38 | |
| Mean (SD) | 0.356 | (0.858) | 0.383 | (0.992) | 0.167 | (0.465) |

| | | | |
|---|---|---|---|
| Median | 0.000 | 0.000 | 0.000 |
| Min:Max | 0.00:4.12 | 0.00:4.62 | 0.00:2.16 |

^{a}The total number of confirmed relapses that occurred during the study divided by the total number of patient-years followed in the study.
^{b}Poisson model with the total number of confirmed relapses onset between randomization date and last dose date as the response variable, treatment, IFN-beta dose stratum, and region as covariates, and log-transformed standardized study duration as an offset variable.
^{c}The number of confirmed relapses for each patient divided by the number of years followed in the study for that patient.

We claim:

1. A method for treating relapsing-remitting form of multiple sclerosis, in a patient in need thereof, comprising administering to the patient about 7 mg or about 14 mg teriflunomide, and a pharmaceutically effective amount of interferon beta-1a or 1b.

2. The method according to claim 1, comprising administering to the patient about 7 mg or about 14 mg teriflunomide once a day, and a stable dose of interferon beta-1a or 1b.

3. The method according to claim 2, which is clinically proven effective.

4. A method for reducing the number of T1-Gd lesions in a patient afflicted with relapsing-remitting form of multiple sclerosis comprising administering to the patient about 7 mg or about 14 mg teriflunomide, and a pharmaceutically effective amount of interferon beta-1a or 1b.

5. The method according to claim 4, comprising administering to the patient about 7 mg or about 14 mg teriflunomide once a day, and a stable dose of interferon beta-1a or 1b.

6. The method according to claim 5, which is clinically proven effective.

7. The method according to claim 5, wherein the method reduces more T1-Gd lesions in the patient than when the patient is administered a stable dose of interferon beta-1a or 1b alone.

8. The method according to claim 7, which is clinically proven effective.

9. The method according to claim 5, which comprises administering to the patient about 7 mg teriflunomide once a day, and a stable dose of interferon beta-1a or 1b, wherein the number of T1-Gd lesions in the patients is reduced about 82.6% to about 84.6% comparing to the number of lesions in patients treated by a stable dose of interferon beta-1a or 1b alone.

10. The method according to claim 5, which comprises administering to the patient about 14 mg teriflunomide once a day, and a stable dose of interferon beta-1a or 1b, wherein the number of T1-Gd lesions in the patients is reduced about 82.8% to about 84.4% comparing to the number of lesions in patients treated by a stable dose of interferon beta-1a or 1b alone.

11. A method for reducing the volume of T1-Gd lesions in a patient afflicted with relapsing-remitting form of multiple sclerosis, comprising administering to the patient about 7 mg or about 14 mg teriflunomide once a day, and a stable dose of interferon beta-1a or 1b.

12. The method according to claim 11, which is clinically proven effective.

13. The method according to claim 11, wherein more volume of T1-Gd lesions is reduced in the patient treated by the method than in a patient treated by a stable dose of interferon beta-1a or 1b alone.

14. The method according to claim 13, which comprises administering to the patients about 14 mg teriflunomide once a day, and a stable dose of interferon beta-1a or 1b, wherein more volumes of T1-Gd lesions are reduced in the patients than in patients treated by a stable dose of interferon beta-1a or 1b alone, and wherein the method is clinically proven effective.

15. The method according to claim 11, which comprises administering to the patient about 14 mg teriflunomide once a day, and a stable dose of interferon beta-1a or 1b, wherein the volume of T1-Gd lesions in the patients treated by the method is reduced about 64.7% to about 70.6% comparing to the volume of lesions in patients treated by a stable dose of interferon beta-1a or 1b alone.
